(19) United States
(12) Patent Application Publication
(10) Pub. No.: US 2018/0311658 A1
(43) Pub. Date: Nov. 1, 2018

(54) Catalysts Prepared from Nanostructures of MnO2 and WO3 for Oxidative Coupling of Methane

(71) Applicants: Wugeng LIANG, Richmond, TX (US); Vidya Sagar Reddy SARSANI, Pearland, TX (US); David WEST, Bellaire, TX (US); Aghaddin MAMEDOV, Sugar Land, TX (US); James LOWREY, Pearland, TX (US); Istvan LENGYEL, Lake Jackson, TX (US); Sabic Global Technologies, B.V., BERGEN OP ZOOM (NL)

(72) Inventors: Wugeng LIANG, Richmond, TX (US); Vidya Sagar Reddy SARSANI, Pearland, TX (US); David WEST, Bellaire, TX (US); Aghaddin MAMEDOV, Sugar Land, TX (US); James LOWREY, Pearland, TX (US); Istvan LENGYEL, Lake Jackson, TX (US)

(21) Appl. No.: 15/770,957

(22) PCT Filed: Sep. 14, 2016
(86) PCT No.: PCT/US2016/051623

§ 371 Date: Apr. 25, 2018

Related U.S. Application Data
(60) Provisional application No. 62/246,906, filed on Oct. 27, 2015.

(51) Int. Cl. B01J 37/03; B01J 37/04; B01J 37/08; B01J 37/34; B01J 35/00; B01J 35/02; B01J 21/08; B01J 23/00; C07C 2/84

(52) U.S. Cl. CPC B01J 37/033; B01J 37/04; B01J 37/08; B01J 37/343; B01J 35/0013; B01J 35/0006; B01J 2523/72; B01J 35/026; B01J 21/08; B01J 23/002; C07C 2/84; B01J 2523/12; B01J 2523/69; B01J 35/023

(57) ABSTRACT

Disclosed is a process to prepare a [MnNaW]Oₙ/SiO₂ catalyst using manganese oxide (MnO₂) and tungsten oxide (WO₃) nanostructures. Also disclosed are methods and systems using the aforementioned catalyst having higher methane conversion and C₂ to C₄ selectivity compared to similar catalysts not prepared with MnO₂ and WO₃ nanostructures.

### CROSS REFERENCE TO RELATED APPLICATIONS

This application is a filing under 35 U.S.C. 371 of International Application No. PCT/US2016/051623 filed Sep. 14, 2016, entitled “Catalysts Prepared From Nanostructures of MnO₂ And WO₃ For Oxidative Coupling Of Methane”, which claims priority to U.S. Provisional Application No. 62/246,906 field Oct. 27, 2015, which applications are incorporated by reference herein in their entirety.

### BACKGROUND OF THE INVENTION

### Field of the Invention

The invention generally concerns methods for preparing and using catalysts in the oxidative coupling of methane reaction. In particular, a [MnNaW]Oₙ/SiO₂ catalyst can be prepared by heat treatment of a mixture that include manganese oxide (MnO₂) nanostructures, tungsten oxide (WO₃) nanostructures, silica sol, and a sodium source. The resulting catalyst can be used in the production of C₂ to C₄ hydrocarbons from methane with higher methane conversion and C₂ to C₄ selectivity when compared with catalysts that were produced with MnO₂ and WO₃ microstructures.

### Description of Related Art

Ethylene is the world's largest commodity chemical and the chemical industry's fundamental building block. For example, ethylene derivatives are typically found in food packaging, eyeglasses, cars, medical devices, lubricants, engine coolants and liquid crystal displays. For industrial scale applications ethylene is currently produced by heating natural gas condensates and petroleum distillates, which include ethane and higher hydrocarbons, and the produced ethylene is separated from the product mixture using gas separation processes.

Ethylene can also be produced by oxidative coupling of methane (OCM). One caveat of OCM is that the four strong tetrahedral C—H bonds (435 kJ/mol) of methane offer no functional group, magnetic moments or polar distributions to undergo chemical attack. This makes methane less reactive than nearly all of its conversion products, limiting efficient utilization of natural gas, the world's most abundant petrochemical resource. In the reaction, methane is activated on the catalyst surface at high temperatures, presumably forming methyl radicals which then couple in the gas phase to form ethane, followed by dehydrogenation to ethylene. Additionally, the OCM reaction of methane is exothermic and this exothermicity can lead to a further increase of catalyst bed temperature and uncontrolled heat excursions that can produce agglomeration on the catalyst. The result from these reactions is usually catalyst deactivation and a further decrease in ethylene selectivity. Furthermore, the produced ethylene is highly reactive and can form unwanted and thermodynamically favored oxidation products at higher oxygen concentrations. For example, non-selective over-oxidation of hydrocarbons to CO and CO₂ (e.g., complete oxidation) is a major competing side reaction. Other undesirable products, such as methanol and formaldehyde have also been observed that rapidly react to form CO and CO₂.

Over the past 30 years since the first reported OCM reaction, many methane activation catalysts have been developed. Among these catalysts, Mn—Na₂WO₄ supported on silicon dioxide (SiO₂) is one of the very few catalysts for OCM whose stability over extended times on stream has been reported by several research groups. Overall Mn—Na₂WO₄/SiO₂ shows good conversion, selectivity and single pass yield. Mn—Na₂WO₄/SiO₂ was first reported in 1992 (X. P. Fang, et al., Journal of Molecular Catalysis (China) vol 6 (1992) 255-262 and 427-433) and, as shown in recent reviews (Arndt S., et al., Applied Catalysis A: General, 425-426 (2012) 53-61), there has not been any substantial improvements since it was first introduced as a catalyst for OCM. In general, few supported conventional OCM catalysts have been able to exceed 20-25% combined C₂+ hydrocarbon yield (i.e. ethane and ethylene), and all higher conversion and selectivity are reported at high temperatures where catalyst stability is low. For example, the temperature required to achieve optimum performance of a Mn—Na₂WO₄/SiO₂ system is often 800° C. or above. At this temperature, the catalyst can also have a higher liquid phase to solid phase ratio that promotes undesirable catalyst vaporization. As discussed, a highly exothermic reaction requiring high reaction temperatures has many disadvantages, including catalyst deactivation which results in shorting the life or reactivity of the catalyst.

Several recent disclosures have focused on the use of nanowires in combination with biological templates such as bacteriophages, amyloid fibers, viruses, and capsids in an attempt to produce catalysts for running the OCM reaction. By way of example, U.S. Patent Publication No. 2012/0041246 discloses the use of Li/MgO nanowires, MgNaLa₂O₃ nanowires, La₂O₃ nanowires obtained from biological templates for the use in an OCM reaction. Similarly, U.S. Pat. No. 8,962,517 discloses the use of various magnesium, barium, calcium, strontium oxide, carbonate, sulfate, phosphate, and aluminate nanowires obtained from biological templates for use in an OCM reaction. Also, U.S. Patent Publication No. 2013/0165728 discloses a method of making a methane oxidative coupling catalyst by using nano wire mixed oxides obtained from a biological template. The use of biological templates, however, is expensive and inefficient for commercial manufacture. Further, it introduces additional complexities in the manufacturing process.

### SUMMARY OF THE INVENTION

A solution to the above described problems for catalysts used in oxidative coupling of methane (OCM) reactions has been discovered. The solution resides in a [MnNaW]Oₙ/SiO₂ catalyst prepared by a simplified method using nanostructures of manganese oxide (MnO₂) and tungsten oxide (WO₃). The MnO₂ and WO₃ nanostructures are used in combination with a silica sol solution having a sodium source to form a mixture, which is then dried to obtain a crystalline material and calcined to produce the [MnNaW]Oₙ/SiO₂ catalysts of the present invention. This process is cost efficient and scalable for commercial production. Notably, the use of biological templates such as such as bacteriophages, amyloid fibers, viruses, and capsids are not required. Further, the produced catalysts have been shown in non-limiting embodiments (see Examples) to have a higher methane conversion and light olefin (C₂-C₄) selectivity when compared with a [MnNaW]Oₙ/SiO₂ catalyst prepared in the same manner from MnO₂ and WO₃ microstructures. The improved conversion and selectivity of the catalysts can be maintained even at lower reaction temperatures (e.g., equal to or less than 800° C., between 650° C. to less than 800° C., or from 675° C. to 725° C.), thereby improving the OCM reaction efficiency and catalyst lifespan, while also lowering the production of undesired oxidation byproducts. Without wishing to be bound by theory, it is believed that the use of MnO₂ and WO₃ nanostructures during the production process allows for a more efficient dispersion of these materials in the resulting catalyst, thereby increasing catalytic performance through an increase in the available surface area of the MnO₂ and WO₃ nanostructures to react with reactants. It was further discovered that by using a sonication step during the production process, greater dispersion of the MnO₂ and WO₃ nanostructures can be obtained, thereby producing an even more efficient OCM catalyst.

In one aspect of the invention, there is disclosed a [MnNaW]Oₙ/SiO₂ catalyst capable of catalyzing an oxidative coupling of methane (OCM) reaction and methods for producing the catalyst. By way of example, the catalysts of the present invention can be prepared by (1) obtaining a mixture including manganese oxide (MnO₂) nanostructures, tungsten oxide (WO₃) nanostructures, silica sol, and a sodium source, wherein the MnO₂ nanostructures and WO₃ nanostructures are dispersed throughout the mixture, (2) drying the mixture to obtain a crystalline material and (3) calcining the crystalline material to obtain a [MnNaW]Oₙ/SiO₂ catalyst, wherein n equals the combined valence states of Mn, Na, and W. A [MnNaW]Oₙ/SiO₂ catalyst prepared by this method surprisingly has greater methane conversion and C₂+ hydrocarbon selectivity as compared to a [MnNaW]Oₙ/SiO₂ catalyst that has been prepared in the same manner from MnO₂ and WO₃ powders. In a particular aspect, the MnO₂ nanostructures and WO₃ nanostructures are each individually nanowires, nanoparticles, nanorods, nanotubes, nanocubes, or a combination thereof and are obtained from a hydrothermal process. The sodium source can be NaNO₃, Na₂CO₃, NaCl, or Na₂O, or a mixture thereof. The mixture that includes the MnO₂ nanostructures, WO₃ nanostructures, silica sol, and a sodium source can be obtained by: combining an aqueous dispersion of nanostructures (e.g., MnO₂ and/or WO₃ nanostructures) with a silica sol. An aqueous solution of sodium nitrate (NaNO₃) can be added to the nanostructure-silica sol mixture and the mixture can be agitated at about 80° C. for a desired amount of time (e.g., 2 to 4 hours). In some aspects, the mixing of the mixture includes sonicating or ultrasonicating the mixture for about 2 hours. The mixture includes can be dried at a temperature of 110° C. to 125° C. for 1 hours to 15 hours to obtain a crystalline material, which can be calcined in the presence of air at a temperature of 600° C. to 1000° C. for 5 hours to 10 hours. In other aspects of the current invention, the hydrothermal method consists essentially of or consists of obtaining a mixture comprising a manganese oxide (MnO₂) nanostructures, tungsten oxide (WO₃) nanostructures, silica sol, and a sodium source, wherein the MnO₂ nanostructures and WO₃ nanostructures are dispersed throughout the mixture; drying the mixture to obtain a crystalline material; and calcining the crystalline material to obtain a [MnNaW]Oₙ/SiO₂ catalyst, wherein n equals the combined valence states of Mn, Na, and W. In particular, a template or biological template is not used to prepare the MnO₂ nanostructures, the WO₃ nanostructures, and/or the [MnNaW]Oₙ/SiO₂ catalyst.

Also disclosed is a method for producing C₂+ hydrocarbons from an oxidative coupling of methane reaction. The method involves contacting a reactant feed that includes methane (CH₄) and oxygen (O₂) with a [MnNaW]Oₙ/SiO₂ catalyst under reaction conditions sufficient to produce a product stream comprising C₂+ hydrocarbons, wherein n equals the combined valence states of Mn, Na, and W, and wherein the [MnNaW]Oₙ/SiO₂ catalyst is the product of thermal treatment of a mixture comprising manganese oxide (MnO₂) nanostructures, tungsten oxide (WO₃) nanostructures, silica sol, and a sodium source, wherein the MnO₂ and WO₃ nanostructures are dispersed throughout the mixture. The above mentioned thermal treatment includes heating the mixture to obtain a crystalline material and calcining the crystalline material to obtain the catalyst. The selectivity of C₂+ hydrocarbons during this process can be 55% to 80% at a reaction temperature of 650° C. to 800° C. and a CH₄/O₂ reactant feed ratio of 7.4. The CH₄ conversion can be 5% to 20%, and the O₂ conversion can be 80% to 100% at a reaction temperature of 650° C. to 800° C. In some aspects, the reaction occurs in a continuous flow reactor. The continuous flow reactor can be a fixed-bed reactor, a fluidized reactor, or a moving bed reactor. The method can further include collecting or storing the product stream comprising C₂+ hydrocarbons and using the product stream comprising C₂+ hydrocarbons to produce a petrochemical or a polymer.

In a further embodiment of the present invention there is disclosed a system for producing C₂+ hydrocarbons from an oxidative coupling of methane reaction. The system can include an inlet for a reactant feed containing methane (CH₄) and oxygen (O₂), a reaction zone that can be configured to be in fluid communication with the inlet. The reaction zone can contain the [MnNaW]Oₙ/SiO₂ catalyst detailed above. An outlet can be configured to be in fluid communication with the reaction zone and configured to remove a first product stream including C₂+ hydrocarbons from the reaction zone. The reaction zone can further include the reactant feed and the first product stream. The temperature of the reactant zone can be 600° C. to 900° C., 625° C. to 875° C., or about 630° C. to 850° C., or 675° C. to 725° C. The reaction zone can be a continuous flow reactor selected from a fixed-bed reactor, a fluidized reactor, or a moving bed reactor.

The term “catalyst” means a substance which alters the rate of a chemical reaction. A catalyst may either increase the chemical reaction rate (i.e., a “positive catalyst”) or decrease the reaction rate (i.e., a “negative catalyst”). Catalysts participate in a reaction in a cyclic fashion such that the catalyst is cyclically regenerated. “Catalytic” means having the properties of a catalyst.

The term “conversion” means the mole fraction (i.e., percent) of a reactant converted to a product or products.

The term “selectivity” refers to the percent of converted reactant that went to a specified product, e.g., C₂+ hydrocarbon selectivity is the % of methane that formed ethane, ethylene and higher hydrocarbons.

“Nanostructure” refers to an object or material in which at least one dimension of the object or material is equal to or less than 1000 nm (e.g., one dimension is 1 to 1000 nm in size). In a particular aspect, the nanostructure includes at least two dimensions that are equal to or less than 1000 nm (e.g., a first dimension is 1 to 1000 nm in size and a second dimension is 1 to 1000 nm in size). In another aspect, the nanostructure includes three dimensions that are equal to or less than 1000 nm (e.g., a first dimension is 1 to 1000 nm in size, a second dimension is 1 to 1000 nm in size, and a third dimension is 1 to 1000 nm in size). The shape of the nanostructure can be of a wire, a particle, a sphere, a rod, a tetrapod, a hyper-branched structure, a tube, a cube, or mixtures thereof.

“Nanoparticles” include particles having an average diameter size of 1 and 1000 nanometers.

“Microstructure” refers to an object or material in which at least one dimension of the object or material is greater than 1000 nm (e.g., 1005 nm up to 5000 nm) and in which no dimension of the structure is 1000 nm or smaller.

The term “template” means any synthetic and/or natural material that provides at least one nucleation site where ions can nucleate and grow to form nanostructures. A “biological template” includes biological organic materials that have at least one binding site(s) that recognize certain ions and allow for the nucleation and growth of the same. Non-limiting examples of biological templates include bacteriophages, amyloid fibers, viruses, and capsids.

The terms “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The processes and catalysts of the present invention can “comprise,” “consist essentially of,” or “consist of” particular process steps, ingredients, components, compositions, etc. disclosed throughout the specification. With respect to the transitional phase “consisting essentially of,” in one non-limiting aspect, a basic and novel characteristic of the processes of the present invention is the ability to produce [MnNaW]Oₙ/SiO₂ catalysts from a mixture comprising MnO₂ and WO₃ nanostructures, a silica sol, and a sodium source by heating the mixture to obtain a crystalline material, and calcining the crystalline material to produce the catalysts.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 is a flow diagram showing the procedure for the preparation of [MnNaW]Oₙ/SiO₂ catalysts of the present invention.

FIG. 2 is a schematic of a system of the present invention that include the [MnNaW]Oₙ/SiO₂ capable of catalyzing the oxidative coupling of methane.

FIG. 3 shows a graphical illustrations comparing methane conversion at different reaction temperatures between [MnNaW]Oₙ/SiO₂ prepared with nanostructures of MnO₂ and WO₃ and micronized powder of MnO₂ and WO₃.

FIG. 4 shows a graphical illustrations comparing oxygen conversion at different reaction temperatures between [MnNaW]Oₙ/SiO₂ prepared with nanostructures of MnO₂ and WO₃ and micronized powder of MnO₂ and WO₃.

FIG. 5 shows a graphical illustrations comparing C₂+ selectivities at different reaction temperatures between [MnNaW]Oₙ/SiO₂ with nanostructures of MnO₂ and WO₃ and micronized powder of MnO₂ and WO₃.

### DETAILED DESCRIPTION OF THE INVENTION

In order to achieve commercially viable conversion and selectivities in the oxidative coupling of methane (OCM) reaction to produce light olefins (C₂ to C₄ hydrocarbons) often require high reactions temperatures (>800° C.). At these temperatures catalyst deactivation from agglomeration of material on the catalyst surface (coking) and control of runaway heat from the exothermic reaction between oxygen and methane is problematic. The effect is inefficient ethylene production as well as associated increased production costs.

A discovery has been made where manganese oxide (MnO₂) and tungsten oxide (WO₃) nanostructures prepared by a hydrothermal process can be used to provide a [MnNaW]Oₙ/SiO₂ catalyst useful for the production of C₂ to C₄ hydrocarbons from methane with higher methane conversion and C₂ to C₄ selectivity compared to similar catalysts prepared from powder MnO₂ and WO₃. The current invention provides methods and systems that allow a lowered operating temperature of the OCM reaction (<800° C.), thereby permitting improved catalyst stability and selectivity and reducing the production of undesired oxidation byproducts.

These and other non-limiting aspects of the present invention are discussed in further detail in the following sections.

The catalysts of the present invention include catalytic material and an underlying support. The catalytic material can include a manganese, sodium, tungsten, and oxygen having general formula [MnNaW]Oₙ wherein n equals the combined valence states of Mn, Na, and W. Not to be limited by theory, the catalytic material can be one or more of [MnNaW]Oₙ, [MnNaW]O₂, [MnNaW]O₃, [MnNaW]O₄, [MnNaW]O₅, [MnNaW]O₆, [MnNaW]O₇, [MnNaW]O₈, [MnNaW]O₉, Mn/Na₂WO₄, Na/Mn/O, Mn₂O₃/Na₂WO₄, Mn₃O₄/Na₂WO₄, MnWO₄/Na₂WO₄, MnWO₄/Na₂WO₄, Mn/WO₄, Na₂WO₄/Mn, or any combination thereof. The catalytic material disclosed herein can be prepared from a mixture of manganese oxide (MnO₂) nanostructures, tungsten oxide (WO₃) nanostructures, silica sol, and a sodium source.

Materials with structure at the nanoscale level often have unique optical, electronic, or mechanical properties. These properties can vary on the size and shape of the nanostructure and can be further tuned by modification of those synthetic technics used in their preparation, such as precipitation, impregnation, plasma arc gas condensation, wire explosion, sol-gel, chemical templating, or chemical vapor deposition (CVD). The MnO₂ and WO₃ nanostructures used to prepare to the catalytic material of the current invention can be each individually nanowires, nanoparticles, nanorods, nanotubes, nanocubes, or a combination thereof. In a particular aspect, the MnO₂ and WO₃ nanostructures are prepared by a hydrothermal process. The hydrothermal process can include techniques of crystallizing the material from high-temperature aqueous solutions at high vapor pressures. Crystal growth can performed in a pressure vessel, such as an autoclave, by a temperature-difference method, temperature-reduction method, or a metastable-phase technique. The MnO₂ can have dimensions of 10 nm×10 microns. The WO₃ can have dimensions of 20 nm×20 microns. The disclosed hydrothermal process provides a cost efficient access to MnO₂ and WO₃ nanostructures in comparison to alternative nanotechnological processes. In a preferred aspect, a template or biological template is not used to prepare the manganese oxide (MnO₂) nanostructures or the tungsten oxide (WO₃) nanostructures.

The sodium (Na) source for the catalytic material can be any material that contains a sodium cation where the corresponding anionic component does not desolvate, complex, react, or otherwise interfere, alter, or prevent the formation of the catalyts. Preferably the sodium source is NaNO₃, Na₂CO₃, NaCl, or Na₂O, or a mixture thereof. A non-limiting example of a commercial source of the above mentioned sodium sources is Sigma Aldrich® (U.S.A).

The support material or a carrier can be porous and have a high surface area. In some embodiments, the support is active (i.e., has catalytic activity). In other aspects, the support is inactive (i.e., non-catalytic). The support can be an inorganic oxide. In some embodiments, the support includes an inorganic oxide, alpha, beta or theta alumina (Al₂O₃), activated Al₂O₃, silicon dioxide (SiO₂), titanium dioxide (TiO₂), magnesium oxide (MgO), calcium oxide (CaO), strontium oxide (SrO), zirconium oxide (ZrO₂), zinc oxide (ZnO), lithium aluminum oxide (LiAlO₂), magnesium aluminum oxide (MgAlO₄), manganese oxides (MnO, MnO₂, Mn₂O₄), lantheum oxide (La₂O₃), activated carbon, silica gel, zeolites, activated clays, silicon carbide (SiC), diatomaceous earth, magnesia, aluminosilicates, calcium aluminate, or combinations thereof. Preferably the support material is SiO₂ and the source of SiO₂ is silica sol. Silica sol fining agent, for example BEVASIL® 30 (AkzoNobel, USA) or LEVASIL® 200/30% FG (AkzoNobel, USA), are transparent, slightly opalescent aqueous silicic acid solutions and contain 30% colloidal SiO₂. SiO₂ is typically a water insoluble and amorphous solid but can be prepared in aqueous solution by colloid distribution. In a preferred aspect, the catalytic material and support has general formula [MnNaW]Oₙ/SiO₂ wherein n equals the combined valence states of Mn, Na, and W. Not to be limited by theory, the supported catalyst can be one or more of [MnNaW]Oₙ/SiO₂, [MnNaW]O₂/SiO₂, [MnNaW]O₃/SiO₂, [MnNaW]O₄/SiO₂, [MnNaW]O₅/SiO₂, [MnNaW]O₆/SiO₂, [MnNaW]O₇/SiO₂, [MnNaW]O₈/SiO₂, [MnNaW]O₉/SiO₂.

All of the materials used to make the supported catalysts of the present invention can be purchased or made by processes known to those of ordinary skill in the art (e.g., precipitation/co-precipitation, sol-gel, templates/surface derivatized metal oxides synthesis, solid-state synthesis, of mixed metal oxides, microemulsion technique, solvothermal, sonochemical, combustion synthesis, etc.).

The amount of catalytic metal on the support material depends, inter alia, on the catalytic activity of the catalyst. In some embodiments, the amount of catalyst present on the support ranges from 1 to 100 parts by weight of catalyst per 100 parts by weight of support or from 10 to 50 parts by weight of catalyst per 100 parts by weight of support. In other embodiments, the amount of catalyst present on the support ranges from 100-200 parts of catalyst per 100 parts by weight of support, or 200-500 parts of catalyst per 100 parts by weight of support, or 500-1000 parts of catalyst per 100 parts by weight of support material.

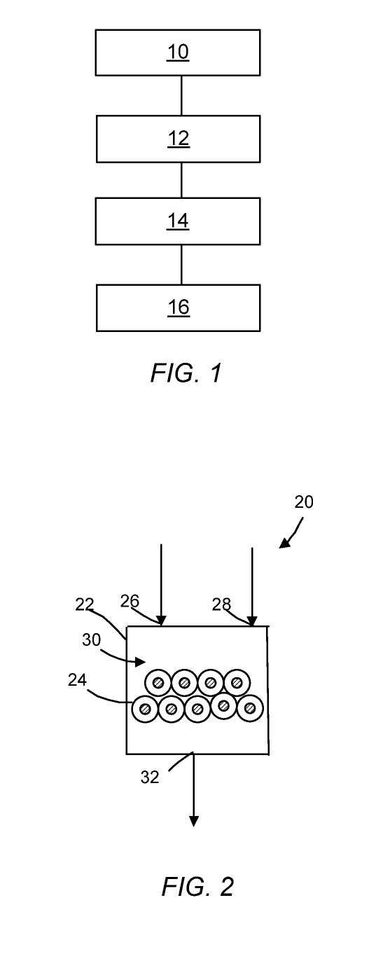

Supported catalysts may be prepared using generally known catalyst preparation techniques. Herein, the support material can be blended with the catalytic material to make a catalytic mixture, which can then be dried to form a crystalline material. By way of example, a method for preparing a [MnNaW]Oₙ/SiO₂ catalyst can include obtaining a mixture including manganese oxide (MnO₂) nanostructures, tungsten oxide (WO₃) nanostructures, silica sol, and a sodium source. The MnO₂ nanostructures and WO₃ nanostructures can be dispersed throughout the mixture. The mixture can be dried to obtain a crystalline material, which is then calcined and to obtain a [MnNaW]Oₙ/SiO₂ catalyst, wherein n equals the combined valence states of Mn, Na, and W. FIG. 1 is a flowchart for the method of preparing the [MnNaW]Oₙ/SiO₂ catalyst. Referring to FIG. 1, in step 10, nano MnO₂, nano WO₃, silica sol, and NaNO₃ are obtained. In step 12, the catalytic material and support may be mixed with suitable mixing equipment to form a catalytic material/support mixture. In some embodiments, the nano MnO₂ and the nano WO₃ can be added together or individually to the silica sol or a silica sol/nanostructure mixture. In other embodiments, the silica sol is added to the either an aqueous solution of the nano MnO₂ and/or the nano WO₃. It should be understood that the order of addition can occur in any manner. Examples of suitable mixing equipment include tumblers, stationary shells or troughs, Muller mixers (for example, batch type or continuous type), impact mixers, sonicators, and any other generally known mixer, or generally known device, that will suitably provide the catalytic material/support mixture. In certain embodiments, the mixing comprises sonicating or ultrasonicating the mixture. The materials can be mixed with heat or without heat until the catalytic material is substantially homogeneously dispersed in the support. In some embodiments, the materials are mixed at 75 to 90° C. for 1 to 5 hours, or 80° C. for 2 hours. After the catalytic material and support are mixed they can be treated thermally. Thermal treatment includes heating the mixture to obtain a crystalline material and calcining the crystalline material. In some embodiments, drying of the mixture to obtain a crystalline material includes subjecting the mixture to a temperature of 110° C. to 125° C. for 1 hours to 15 hours, 120° C. for 12 hours, or until a desired amount of volatile material (e.g., water) is removed from the mixture. In step 16, the crystalline material may be heat treated in the presence of hot air and/or oxygen rich air at a temperature of 600 to 1000° C. for 5 to 10 hours, or 800° C. for 6 hours, to calcine the catalyst (e.g., remove more volatile matter such that at least a portion of the catalytic material is converted to the corresponding metal oxide) and obtain the [MnNaW]Oₙ/SiO₂ catalyst. In a preferred aspect, a template or biological template is not used to prepare the manganese oxide (MnO₂) nanostructures, the tungsten oxide (WO₃) nanostructures, and/or the [MnNaW]Oₙ/SiO₂ catalyst.

The supported catalyst of the present invention includes a dopant or a doping agent or be referred to as being “doped” with metal elements, semi-metal elements, non-metal elements or combinations thereof. In a particular aspect of the invention, the dopant can be or include metallic silver or silver in the form of a salt, for example silver nitrate (AgNO₃). The dopant can be combined with the catalyst by processes known to those of skill in the art (e.g., mixing, precipitation/co-precipitation, impregnation, sol-gel, templates/surface derivatized metal oxides synthesis, solid-state synthesis, of mixed metal oxides, microemulsion technique, solvothermal, sonochemical, combustion synthesis, etc.). The amount of dopant added to the catalyst can range from about 0.01 wt/wt % to about 50 wt/wt %, with all ranges in between, for example from about 0.1 wt/wt % to about 20 wt/wt %, or about 1 wt/wt % to about 10 wt/wt %. In a preferred aspect, the amount of dopant added to the catalyst ranges from about 1 wt/wt % to about 5 wt/wt % and is more specifically added to the catalyst at about 1 wt/wt %, about 2 wt/wt %, about 3 wt/wt %, about 4 wt/wt %, or about 5 wt/wt %.

Additional catalysts can be used in combination with the catalysts of the present invention. The additional catalysts (e.g., a second catalyst, third catalyst, fourth catalyst, etc.) can be positioned up stream or downstream or mixed with the catalysts of the present invention. The additional catalysts can be supported, bulk metal catalysts, or unsupported catalysts. The support can be active or inactive. The catalyst support can include MgO, Al₂O₃, SiO₂, or the like. One or more of the additional catalysts can include one or more metals or metal compounds thereof. Catalytic metals include Li, Na, Ca, Cs, Mg, La, Ce, W, Mn, Ru, Rh, Ni, and Pt. Non-limiting examples of catalysts can include La on a MgO support, Na, Mn, and La₂O₃ on an aluminum support. Non-limiting examples of catalysts that promote oxidative coupling of methane to produce ethylene are Li₂O, Na₂O, Cs₂O, MgO WO₃, Mn₃O₄, or any combination thereof.

The reactant mixture in the context of the present invention can be a gaseous mixture that includes a gaseous hydrocarbon compounds, inert gases, oxygen, steam, or mixtures thereof. The hydrocarbon or mixtures of hydrocarbons can include natural gas, liquefied petroleum gas containing of C₂-C₅ hydrocarbons, C₆+ heavy hydrocarbons (e.g., C₆ to C₂₄ hydrocarbons such as diesel fuel, jet fuel, gasoline, tars, kerosene, etc.), oxygenated hydrocarbons, and/or biodiesel, alcohols, or dimethyl ether. In a preferred aspect, the hydrocarbon is a mixture of hydrocarbons that is predominately methane (e.g., natural gas). The oxygen containing gas used in the present invention can be air, oxygen enriched air, oxygen gas, and can be obtained from various sources. The reactant mixture may further contain other gases or steam, provided that these do not negatively affect the reaction. Examples of such other gases include carbon dioxide, nitrogen and hydrogen. The hydrogen may be from various sources, including streams coming from other chemical processes, like ethane cracking, methanol synthesis, or conversion of methane to aromatics. Carbon dioxide may be from natural gas, or a waste or recycle gas stream (e.g. from a plant on the same site, like for example from ammonia synthesis) or after recovering the carbon dioxide from a gas stream.

The reaction processing conditions in the continuous flow reactor envisioned using the catalyst of the current invention can be varied to achieve a desired result (e.g., C₂+ hydrocarbons product). In one aspect, the process can include contacting a feed stream of hydrocarbon and oxidant with any of the catalysts described throughout the specification under established OCM conditions (e.g., a methane to oxygen ratio of 7.4 and reaction temperature of 725° C.) to afford a methane conversion of greater than 13.4% and a C₂+ selectivity greater than 75.5%. In one aspect of the current invention, the selectivity of C₂+ hydrocarbons is 55% to 80% at a reaction temperature of 650° C. to 800° C. In a particular embodiment, the selectivity of C₂+ hydrocarbons is 55% to 80% at a reaction temperature of 650° C. to 800° C. at a CH₄/O₂ reactant feed ratio of 7.4, at a gas hourly space velocity (GHSV) range from 500 to 100,000 h⁻¹ or more at atmospheric or elevated pressures. In another aspect, the methane conversion of the process is 5% to 20% and the O₂ conversion is 80% to 100% at a reaction temperature of 650° C. to 800° C.

In some aspects, the catalyst of the present invention can be used in continuous flow reactor to produce C₂+ hydrocarbons from methane (e.g., natural gas). Generally, the C₂+ hydrocarbons are obtained from the oxidative coupling of methane. Non-limiting examples of the configuration of the catalytic material in a continuous flow reactor are provided below and throughout this specification. The continuous flow reactor can be a fixed bed reactor, a stacked bed reactor, a fluidized bed reactor, a moving bed reactor, or an ebullating bed reactor. The catalytic material can be arranged in the continuous flow reactor in layers (e.g., catalytic beds) or mixed with the reactant stream (e.g., ebullating bed).

In certain embodiments, a volume of catalyst in the reaction zone of the continuous flow reactor is in a range from about 10-60 vol %, about 20-50 vol %, or about 30-40 vol % of a total volume of reactant in the reaction zone. Processing conditions in the continuous flow reactor may include, but are not limited to, temperature, pressure, oxidant source flow (e.g., air or oxygen), hydrocarbon gas flow (e.g., methane or natural gas), ratio of reactants, or combinations thereof. Process conditions can be controlled to produce C₂+ hydrocarbons with specific properties (e.g., percent ethylene, percent butene, percent butane, etc.). The average temperature of the reaction zone in the continuous flow reactor can be about 600° C., 625° C., 650° C., 655° C., 660° C., 665° C., 670° C., 675° C., 680° C., 685° C., 690° C., 695° C., 700° C., 705° C., 710° C., 715° C., 720° C., 725° C., 730° C., 735° C., 740° C., 745° C., 750° C., 755° C., 760° C., 765° C., 770° C., 775, 780° C., 785° C., 790° C., 795° C., 800° C., 805° C., 810° C., 815° C., 820° C., 825° C. or any value or range there between. Pressure in the continuous flow reactor can range from 0.1 MPa to 1 MPa. The gas hourly space velocity of the reactant feed ranges from 500 h¹ to 50,000 h⁻¹ or more. In some embodiments, the GHSV is as high as can be obtained under the reaction conditions (e.g., 500,000 h⁻¹). In some aspects of the present invention, the reactant mixture can have a molar ratio of methane to oxygen ranges from 0.3 to 20, 0.5 to 15, 1 to 10, or 5 to 7.5 or any range there between. The molar ratio of methane to oxygen can be 0.3, 0.5, 1, 1.5, 2, 2.5, 3, 3.5, 4, 4.5, 5, 5.5, 6, 6.5, 7, 7.5, 8, 8.5, 9, 9.5, 10, 15, or 20 or any value there between. Severity of the process conditions may be manipulated by changing, the hydrocarbon source, oxygen source, pressure, flow rates, the temperature of the process, the catalyst type, and/or catalyst to feed ratio. In a preferred embodiment, the temperature of the reactant zone is 725° C. to 825° C., more preferably 750° C. to 810° C., and most preferably about 800° C. at 1 bara, and/or a gas hourly space velocity (GHSV) as high as can be obtained (e.g., 500,000 h⁻¹).

Referring to FIG. 2, a schematic of system 20 for the production of C₂+ hydrocarbons is depicted. System 2100 may include a continuous flow reactor 22 and a catalytic material 24. In a preferred embodiment, catalytic material 24 is [MnNaW]Oₙ/SiO₂ prepared with nano MnO₂/WO₃ of the present invention. A reactant stream that includes methane can enter the continuous flow reactor 22 via the feed inlet 26. An oxygen containing gas (oxidant) is provided in via oxidant source inlet 28. In some aspects of the invention, methane and the oxygen containing gas are fed to the reactor via one inlet. The reactants can be provided to the continuous flow reactor 22 such that the reactants mix in the reactor to form a reactant mixture prior to contacting the catalytic material 24 in the reaction zone 30. Reaction zone 30 of continuous flow reactor 22 is configured to be in fluid communication with feed inlet 26, oxidant inlet 28, and product outlet 32. In some embodiments, the catalytic material and the reactant feed is heated to the approximately the same temperature. In some instances, the catalytic material 24 may be layered in the continuous flow reactor 22. Contact of the reactant mixture with the catalytic material 24 produces a product stream (for example, C₂+ hydrocarbons and generates heat (i.e., an exotherm or rise in temperature is observed). After contacting the catalyst, the reaction conditions are maintained downstream of the catalytic material at temperatures sufficient to promote continuation of the process. The product stream can exit reaction zone 30 of continuous flow reactor 22 via product outlet 32 which is configured to remove the product stream including C₂+ hydrocarbons from reaction zone 30.

The resulting C₂+ hydrocarbons produced from the systems of the invention are separated using gas/liquid separation techniques, for example, distillation, absorption, membrane technology to produce a gaseous stream that includes carbon monoxide, carbon dioxide, hydrogen, C₂+ hydrocarbons product, and a water stream. The C₂+ hydrocarbons are separated from the hydrogen and carbon monoxide and/or carbon dioxide, if present, using gas/gas separation techniques, for example, a hydrogen selective membrane, a carbon monoxide selective membrane, or cryogenic distillation to produce, C₂+ hydrocarbons, carbon monoxide, carbon dioxide, hydrogen or mixtures thereof. The separated or mixture of products can be used in additional downstream reaction schemes to create additional products or for energy production. Examples of other products include chemical products such as methanol production, olefin synthesis (e.g., via Fischer-Tropsch reaction), aromatics production, carbonylation of methanol, carbonylation of olefins, the reduction of iron oxide in steel production, etc. The method can further include isolating and/or storing the produced gaseous mixture or the separated products.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Comparative Example 1

### Preparation of [MnNaW]Oₙ/SiO₂ Prepared with Powder MnO₂/WO₃

Powdered MnO₂ (0.38 g, −150 microns) wet cake was dispersed into deionized water (10 mL). The aqueous MnO₂ mixture was added to a silica sol (29.35 g, silica content 34%). Powdered WO₃ (0.79 g, −200 microns) was dispersed into deionized water (10 mL) and added to the MnO₂/silica sol mixture. Sodium nitrate (0.58 g, NaNO₃) was dissolved in deionized water (10 mL) and added to the MnO₂/WO₃/silica sol mixture. The mixture was agitated for 2 hours at 80° C., and then dried at 120° C. for 12 hours to obtain a crystalline material. The crystalline material was calcined at 800° C. for 6 hours. The resulting catalyst was sized to a particle size of 35-50 mesh.

### Example 2

### Preparation of [MnNaW]Oₙ/SiO₂ prepared with nano MnO₂/WO₃

MnO₂ wet cake (3.86 g, 10% MnO₂, 10 nm×10 microns) wet cake was dispersed into deionized water (20 mL). The aqueous MnO₂ mixture was added to a silica sol (29.35 g, silica content 34%). WO₃ wet cake (2.92 g, 27% WO₃, 20 nm×20 microns) was dispersed into deionized water (20 mL) and added to the MnO₂/silica sol mixture. Sodium nitrate (0.58 g, NaNO₃) was dissolved in deionized water (10 mL) and added to the MnO₂/WO₃/silica sol mixture. The mixture was agitated for 2 hours at 80° C., and then dried at 120° C. for 12 hours to obtain a crystalline material. The crystalline material was calcined at 800° C. for 6 hours. The resulting catalyst was sized to a particle size of 35-50 mesh.

### Example 3

### Preparation of [MnNaW]₁Oₙ/SiO₂ Prepared with Nano MnO₂/WO₃ and Na₂CO₃

MnO₂ wet cake (3.86 g, 10% MnO₂, 10 nm×10 microns) wet cake was dispersed into deionized water (20 mL). The aqueous MnO₂ mixture was added to a silica sol (29.35 g, silica content 34%). WO₃ wet cake (2.92 g, 27% WO₃, 20 nm×20 microns) was dispersed into deionized water (20 mL) and added to the MnO₂/silica sol mixture. Sodium carbonate (0.36 g, Na₂CO₃) was dissolved in deionized water (10 mL) and added to the MnO₂/WO₃/silica sol mixture. The mixture was agitated for 2 hours at 80° C., and then dried at 120° C. for 12 hours to obtain a crystalline material. The crystalline material was calcined at 800° C. for 6 hours. The resulting catalyst was sized to a particle size of 35-50 mesh.

### Example 4

### Preparation of [MnNaW]Oₙ/SiO₂ Prepared with Nano MnO₂/WO₃ and NaCl

MnO₂ wet cake (3.86 g, 10% MnO₂, 10 nm×10 microns) wet cake was dispersed into deionized water (20 mL). The aqueous MnO₂ mixture was added to a silica sol (29.35 g, silica content 34%). WO₃ wet cake (2.92 g, 27% WO₃, 20 nm×20 microns) was dispersed into deionized water (20 mL) and added to the MnO₂/silica sol mixture. Sodium chloride (0.40 g, NaCl) was dissolved in deionized water (10 mL) and added to the MnO₂/WO₃/silica sol mixture. The mixture was agitated for 2 hours at 80° C., and then dried at 120° C. for 12 hours to obtain a crystalline material. The crystalline material was calcined at 800° C. for 6 hours. The resulting catalyst was sized to a particle size of 35-50 mesh.

### Example 5

### Preparation of [MnNaW]₁Oₙ/SiO₂ prepared with nano MnO₂/WO₃ with sonication

MnO₂ wet cake (3.86 g, 10% MnO₂, 10 nm×10 microns) wet cake was dispersed into deionized water (20 mL). The aqueous MnO₂ mixture was added to a silica sol (29.35 g, silica content 34%). WO₃ wet cake (2.92 g, 27% WO₃, 20 nm×20 microns) was dispersed into deionized water (20 mL) and added to the MnO₂/silica sol mixture. Sodium carbonate (0.36 g, Na₂CO₃) was dissolved in deionized water (10 mL) and added to the MnO₂/WO₃/silica sol mixture. The mixture was sonicated for 2 hours at 80° C., and then dried at 120° C. for 12 hours to obtain a crystalline material. The crystalline material was calcined at 800° C. for 6 hours. The resulting catalyst was sized to a particle size of 35-50 mesh.

### Example 6

### Comparison of Methane (CH₄) and Oxygen (O₂) Conversion Under Different Reaction Temperatures

The catalytic performances of the comparative catalyst of Example 1 and the catalyst of the present invention of Example 2 were compared. A fixed bed catalyst reactor was filled with 100 mg of the catalytic materials of Examples 1-2 with a particle size of 35-50 mesh. The reactor was heated to the required temperature, and a mixture of methane and oxygen at a fixed CH₄:O₂ ratio of 7.4 was fed to the reactor at a total flow rate of 33.3 sccm.

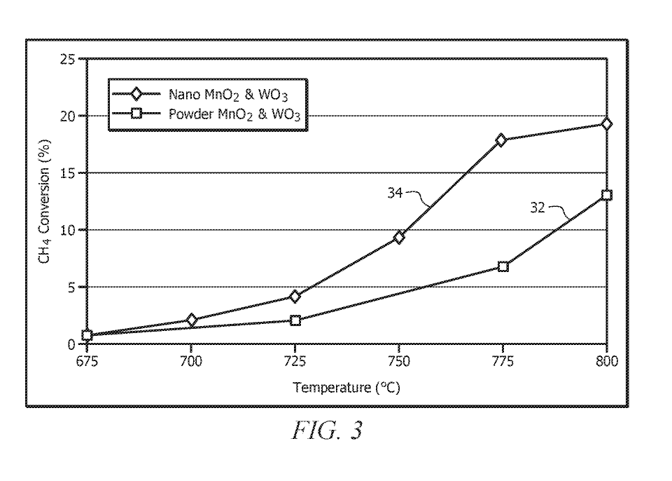

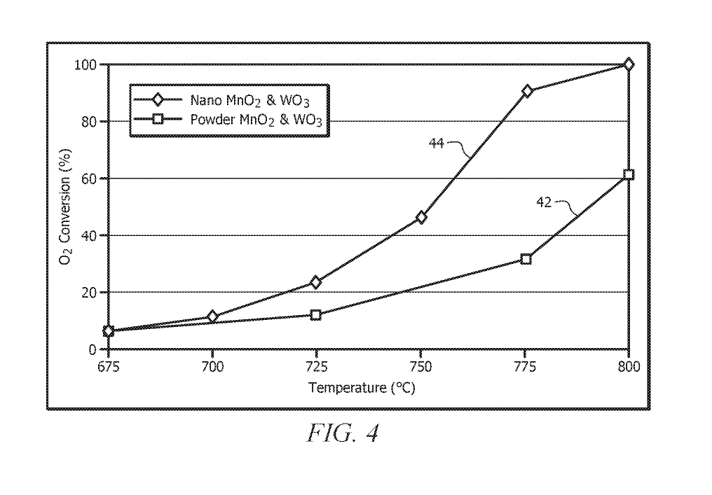

Methane and oxygen conversion was calculated on the basis of the difference of inlet and outlet concentrations of methane. Percent methane conversion for the catalysts from Examples 1 and 2 are shown in FIG. 3. Data line 32 is percent methane conversion using [MnNaW]Oₙ/SiO₂ prepared with powder MnO₂/WO₃. Data line 34 is percent methane conversion using [MnNaW]Oₙ/SiO₂ prepared with nano MnO₂/WO₃. The catalyst prepared using nano MnO₂/WO₃ gave the same methane conversion under a lower temperature, indicating a higher activity in comparison the catalyst prepared with powder MnO₂/WO₃. Percent oxygen conversion for the catalysts from Examples 1 and 2 are shown in FIG. 4. Data line 42 is percent oxygen conversion using [MnNaW]Oₙ/SiO₂ prepared with powder MnO₂/WO₃. Data line 44 is percent oxygen conversion using [MnNaW]Oₙ/SiO₂ prepared with nano MnO₂/WO₃. Similar to the results of methane conversion, the catalyst prepared using nano MnO₂/WO₃ gave the same oxygen conversion under a lower temperature, indicating a higher activity in comparison the catalyst prepared with powder MnO₂/WO₃. To conclude, the use of a nano MnO₂/WO₃ prepared catalyst allows the reaction to be operated under a lower temperature, which helps prevent over-oxidation and catalyst deactivation/vaporization.

### Example 7

### Comparison of C₂+ Selectivity Under Different Reaction Temperatures

A fixed bed catalyst reactor was filled with 100 mg of the catalytic materials of Examples 1-2. The reactor was heated to the required temperature, and a mixture of methane and oxygen at a fixed CH₄:O₂ ratio of 7.4 was fed to the reactor at a total flow rate of 33.3 sccm.

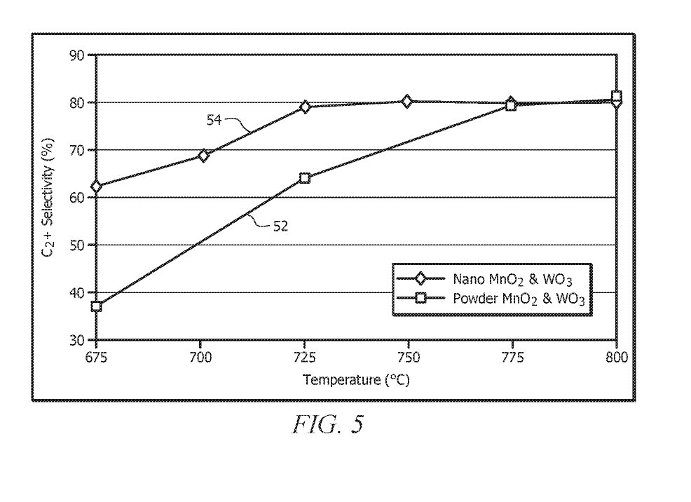

The C₂+ selectivity was calculated on the basis of concentrations of C₂+ products in comparison all the converted amount of methane. C₂+ selectivity for the catalysts from Examples 1 and 2 are shown in FIG. 5. Data line 52 is C₂+ selectivity using [MnNaW]Oₙ/SiO₂ prepared with powder MnO₂/WO₃. Data line 54 is C₂+ selectivity using [MnNaW]Oₙ/SiO₂ prepared with nano MnO₂/WO₃. At lower temperature, the C₂+ selectivity obtained with the catalyst prepared with nano MnO₂/WO₃ was higher than the C₂+ selectivity obtained with the catalyst prepared with powder MnO₂/WO₃. At higher reaction temperatures, the selectivity obtained with these two catalysts were almost the same. Therefore, nano MnO₂/WO₃ prepared catalysts offer higher selectivity over a broaden temperature range than powder MnO₂/WO₃ prepared catalysts. These results provide significant advantages, for example, in a commercial reactor where the temperature profile across axial and radial directions in the catalyst bed can vary as much as 200° C. (See, Lee et al., Fuel, 106 (2013) 851). Therefore, the final selectivity obtained through the commercial reactor is the sum of selectivity contribution from different temperatures. With the selectivity shown in FIG. 5, it is predicted that the final selectivity of a commercial reactor with catalyst prepared with nano MnO₂/WO₃ will be higher than that with a catalyst prepared with powder MnO₂/WO₃.

### Example 8

### Catalytic Performance Comparison with Different Sodium (Na) Sources

Table 1 compares nano MnO₂/WO₃ prepared catalysts (Examples 2-4) made with different sodium reactants with powder MnO₂/WO₃ prepared catalyst (Example 1) and the percent methane and oxygen conversion and the percent C₂+ selectivity under the same testing conditions as in FIG. 3-5 (Examples 6 and 7). The results show that source of the sodium selection is not as significant as switching to nano MnO₂/WO₃ alone, nonetheless NaCl shows the best methane and oxygen conversion and NaNO₃ shows the best C₂+ selectivity.

TABLE 1

| | CH₄ | O₂ | C₂+ |
|---|---|---|---|
| Catalyst Example | Conversion (%) | Conversion (%) | selectivity (%) |
| 1 | 13.1 | 61.9 | 79.1 |
| 2 | 19.4 | 99.2 | 80.1 |
| 3 | 17.4 | 99.9 | 75.7 |
| 4 | 20.3 | 100.0 | 79.1 |

Table 2 compares nano MnO₂/WO₃ prepared catalyst (Example 2) with nano MnO₂/WO₃ prepared catalyst with sonication (Example 5) and the percent methane and oxygen conversion and the percent C₂+ selectivity under the same testing conditions as in FIG. 3-5 (Examples 6 and 7). From the results, it was concluded that sonication improves catalyst dispersion which thereby improves catalyst performance.

TABLE 2

| | CH₄ | O₂ | C₂+ |
|---|---|---|---|
| Catalyst Example | Conversion (%) | Conversion (%) | selectivity (%) |
| 2 | 19.4 | 99.2 | 80.1 |
| 5 | 19.9 | 99.6 | 81.2 |

In summary, the [MnNaW]Oₙ/SiO₂ catalyst prepared with nano MnO₂/WO₃ of the present invention showed higher performance (e.g., higher methane and oxygen conversions and higher C₂+ selectivity), which allows a reactor to be operated under lower temperature which helps prevent over-oxidation and catalyst deactivation/vaporization. Additionally, the same [MnNaW]Oₙ/SiO₂ catalyst prepared with sonication as demonstrated in Table 2 provides C₂+ selectivity up to 81.2%.

### Additional Disclosure

The following enumerated aspects are provided as nonlimiting examples.

A first aspect which is a method for preparing a [MnNaW]Oₙ/SiO₂ catalyst, the method comprising:
(a) obtaining a mixture comprising a manganese oxide (MnO₂) nanostructures, tungsten oxide (WO₃) nanostructures, silica sol, and a sodium source, wherein the MnO₂ nanostructures and WO₃ nanostructures are dispersed throughout the mixture;
(b) drying the mixture to obtain a crystalline material; and
(c) calcining the crystalline material to obtain a [MnNaW]Oₙ/SiO₂ catalyst, wherein n equals the combined valence states of Mn, Na, and W.

A second aspect which is the method of the first aspect, wherein the MnO₂ nanostructures and WO₃ nanostructures are each individually nanowires, nanoparticles, nanorods, nanotubes, nanocubes, or a combination thereof.

A third aspect which is the method of any one of the first to the second aspects, wherein the sodium source is NaNO₃, Na₂CO₃, NaCl, or Na₂O, or a mixture thereof.

A fourth aspect which is the method of any one of the first to the third aspects, wherein the mixture in step (a) is obtained by:
(i) obtaining an aqueous nano MnO₂/silica sol mixture;
(ii) adding an aqueous nano WO₃ mixture to the nano MnO₂/silica sol mixture;
(iii) agitating and heating the MnO₂/nano WO₃ silica sol mixture.

A fifth aspect which is the method of the fourth aspect, wherein mixing comprises sonicating or ultrasonicating the mixture.

A sixth aspect which is the method of any one of the first to the fifth aspects, wherein drying step (b) comprises subjecting the mixture to a temperature of 110° C. to 125° C. for 1 hour to 15 hours.

A seventh aspect which is the method of any one of the first to the sixth aspects, wherein calcining step (c) comprises subjecting the crystalline material to a temperature of 600° C. to 1000° C. for 5 hours to 10 hours.

An eighth aspect which is the method of any one of the first to the seventh aspects, wherein the method consists essentially of or consists of steps (a), (b), and (c).

A ninth aspect which is the method of any one of the first to the seventh aspects, wherein the manganese oxide (MnO₂) nanostructures, the tungsten oxide (WO₃) nanostructures, or both are obtained from a hydrothermal process.

A tenth aspect which is the method of any one of the first to the ninth aspects, wherein a template or biological template is not used to prepare the manganese oxide (MnO₂) nanostructures, the tungsten oxide (WO₃) nanostructures, and/or the [MnNaW]Oₙ/SiO₂ catalyst.

An eleventh aspect which is a [MnNaW]Oₙ/SiO₂ catalyst prepared by the method of any one of the first to the tenth aspects.

A twelfth aspect which is a method for producing C₂+ hydrocarbons from an oxidative coupling of methane reaction, the method comprising contacting a reactant feed that includes methane (CH₄) and oxygen (O₂) with a [MnNaW]Oₙ/SiO₂ catalyst under reaction conditions sufficient to produce a product stream comprising C₂+ hydrocarbons, wherein n equals the combined valence states of Mn, Na, and W, and wherein the [MnNaW]Oₙ/SiO₂ catalyst is the product of thermal treatment of a mixture comprising manganese oxide (MnO₂) nanostructures, tungsten oxide (WO₃) nanostructures, silica sol, and a sodium source, wherein the MnO₂ and WO₃ nanostructures are dispersed throughout the mixture.

A thirteenth aspect which is the method of the twelfth aspect, wherein thermal treatment comprises:
(a) heating the synthesis mixture to obtain a crystalline material; and
(b) calcining the crystalline material.

A fourteenth aspect which is the method of any one of the twelfth to the thirteenth aspects, wherein the MnO₂ nanostructures and WO₃ nanostructures are each individually nanowires, nanoparticles, nanorods, nanotubes, nanocubes, or a combination thereof.

A fifteenth aspect which is the method of any one of the twelfth to the fourteenth aspects, wherein the sodium source is sodium nitrate (NaNO₃), sodium carbonate (Na₂CO₃), sodium chloride (NaCl), or sodium oxide (Na₂O), or a mixture thereof.

A sixteenth aspect which is the method of any one of the twelfth to the fifteenth aspects, wherein the selectivity of C₂+ hydrocarbons is 55% to 80% at a reaction temperature of 675° C. to 800° C. and a CH₄/O₂ reactant feed ratio of 7.4.

A seventeenth aspect which is the method of any one of the twelfth to the sixteenth aspects, wherein the CH₄ conversion is 5% to 20% and the O₂ conversion is 5% to 100% at a reaction temperature of 675° C. to 800° C.

An eighteenth aspect which is the method of any one of the twelfth to the seventeenth aspects, wherein the reaction occurs in a continuous flow reactor.

A nineteenth aspect which is the method of the eighteenth aspect, wherein the continuous flow reactor is a fixed-bed reactor, a fluidized reactor, or a moving bed reactor.

A twentieth aspect which is the method of any one of the twelfth to the nineteenth aspects, further comprising collecting or storing the product stream comprising C₂+ hydrocarbons.

A twenty-first aspect which is the method of any one of the twelfth to the twentieth aspects, further comprising using the product stream comprising C₂+ hydrocarbons to produce a petrochemical or a polymer.

A twenty-second aspect which is a system for producing C₂+ hydrocarbons from an oxidative coupling of methane reaction, the system comprising:
(a) an inlet for a reactant feed comprising methane (CH₄) and oxygen (O₂);
(b) a reaction zone that is configured to be in fluid communication with the inlet, wherein the reaction zone comprises the catalyst of the tenth aspect; and
(c) an outlet configured to be in fluid communication with the reaction zone and configured to remove a product stream comprising C₂+ hydrocarbons from the reaction zone.

A twenty-third aspect which is the system of the twenty-second aspect, wherein the reaction zone further comprises the reactant feed and the first product stream.

A twenty-fourth aspect which is the system of the twenty-third aspect, wherein the temperature of the reactant zone is 600° C. to 900° C., more preferably 750° C. to 810° C., and most preferably about 800° C.

A twenty-fifth aspect which is the system of any one of the twenty-second to the twenty-fourth aspects, wherein the reaction zone is a continuous flow reactor selected from a fixed-bed reactor, a fluidized reactor, or a moving bed reactor.

While various aspects have been shown and described, modifications thereof can be made by one skilled in the art without departing from the spirit and teachings of the disclosure. The aspects described herein are exemplary only, and are not intended to be limiting. Many variations and modifications of the disclosure disclosed herein are possible and are within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present disclosure. The discussion of a reference in the disclosure is not an admission that it is prior art to the present disclosure, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A method for preparing a [MnNaW]Oₙ/SiO₂ catalyst, the method comprising:
(a) contacting manganese oxide (MnO₂) nanostructures (nano MnO₂), tungsten oxide (WO₃) nanostructures (nano WO₃), silica sol, and a sodium source to form a mixture, wherein the MnO₂ nanostructures and WO₃ nanostructures are dispersed throughout the mixture, and wherein a nanostructure has at least one dimension of from 1 nm to 1000 nm;
(b) drying the mixture at a temperature of 110° C. to 125° C. for 1 hour to 15 hours to obtain a crystalline material; and
(c) calcining the crystalline material to obtain a [MnNaW]Oₙ/SiO₂ catalyst, wherein n balances the combined valence states of Mn, Na, and W.

2. The method of claim 1, wherein the MnO₂ nanostructures and WO₃ nanostructures are each individually nanowires, nanoparticles, nanorods, nanotubes, nanocubes, or a combination thereof.

3. The method of claim 1, wherein the sodium source is NaNO₃, Na₂CO₃, NaCl, or Na₂O, or a mixture thereof.

4. The method of claim 1, wherein the mixture in step (a) is obtained by:
(i) contacting the nano MnO₂ and the silica sol to form an aqueous MnO₂ nanostructures/silica sol mixture;
(ii) adding an aqueous nano WO₃ mixture to the aqueous MnO₂ nanostructures/silica sol mixture to form an aqueous MnO₂ nanostructures/WO₃ nanostructures/silica sol mixture; and
(iii) agitating and heating the aqueous MnO₂ nanostructures/WO₃ nanostructures/silica sol mixture.

5. The method of claim 4, wherein agitating comprises sonicating or ultrasonicating the mixture.

6. The method of claim 1, wherein calcining step (c) comprises subjecting the crystalline material to a temperature of 600° C. to 1000° C. for 5 hours to 10 hours.

7. The method of claim 1, wherein the manganese oxide (MnO₂) nanostructures, the tungsten oxide (WO₃) nanostructures, or both are obtained from a hydrothermal process.

8. A [MnNaW]Oₙ/SiO₂ catalyst comprising a thermally treated mixture, wherein the mixture comprises manganese oxide (MnO₂) nanostructures, tungsten oxide (WO₃) nanostructures, silica sol, and a sodium source, wherein the MnO₂ nanostructures and WO₃ nanostructures are dispersed throughout the mixture, and wherein a nanostructure has at least one dimension of from 1 nm to 1000 nm.

9. The [MnNaW]Oₙ/SiO₂ catalyst of claim 8 prepared by the method of claim 1.

10. A method for producing C₂+ hydrocarbons from an oxidative coupling of methane reaction, the method comprising contacting a reactant feed that includes methane (CH₄) and oxygen (O₂) with a [MnNaW]Oₙ/SiO₂ catalyst at a reaction temperature of from 600° C. to 900° C. to produce a product stream comprising C₂+ hydrocarbons, wherein n balances the combined valence states of Mn, Na, and W, and wherein the [MnNaW]Oₙ/SiO₂ catalyst comprises a thermally treated mixture, wherein the mixture comprises manganese oxide (MnO₂) nanostructures, tungsten oxide (WO₃) nanostructures, silica sol, and a sodium source, wherein the MnO₂ nanostructures and WO₃ nanostructures are dispersed throughout the mixture, and wherein a nanostructure has at least one dimension of from 1 nm to 1000 nm.

11. The method of claim 10, wherein thermal treatment comprises:
(a) heating the mixture to obtain a crystalline material; and
(b) calcining the crystalline material.

12. The method of claim 10, wherein the MnO₂ nanostructures and WO₃ nanostructures are each individually nanowires, nanoparticles, nanorods, nanotubes, nanocubes, or a combination thereof; and wherein the sodium source is sodium nitrate (NaNO₃), sodium carbonate (Na₂CO₃), sodium chloride (NaCl), or sodium oxide (Na₂O), or a mixture thereof.

13. The method of claim 10, wherein the selectivity of C₂+ hydrocarbons is 55% to 80% at a reaction temperature of 675° C. to 800° C. and a CH₄/O₂ reactant feed ratio of 74.

14. The method of claim 10, wherein the CH₄ conversion is 5% to 20% and the O₂ conversion is 5% to 100% at a reaction temperature of 675° C. to 800° C.

15. The method of claim 10, wherein the reaction occurs in a continuous flow reactor.

16. The method of claim 15, wherein the continuous flow reactor is a fixed-bed reactor, a fluidized reactor, or a moving bed reactor.

17. A system for producing C₂+ hydrocarbons from an oxidative coupling of methane reaction, the system comprising:
(a) an inlet for a reactant feed comprising methane (CH₄) and oxygen (O₂);
(b) a reaction zone that is configured to be in fluid communication with the inlet, wherein the reaction zone comprises the catalyst of claim 8; and
(c) an outlet configured to be in fluid communication with the reaction zone and configured to remove a product stream comprising C₂+ hydrocarbons from the reaction zone.

18. The system of claim 17, wherein the reaction zone further comprises the reactant feed and the first product stream.

19. The system of claim 18, wherein the temperature of the reactant zone is 600° C. to 900° C.

20. The system of claim 17, wherein the reaction zone is a continuous flow reactor selected from a fixed-bed reactor, a fluidized reactor, or a moving bed reactor.

21. The method of claim 10, wherein the step (a) of heating the mixture to obtain a crystalline material comprises subjecting the mixture to a temperature of 110° C. to 125° C. for 1 hour to 15 hours.
